(19) United States
(12) Patent Application Publication
(10) Pub. No.: US 2009/0024173 A1
(43) Pub. Date: Jan. 22, 2009

(54) Bone Plate

(76) Inventor: Geraldo Marins dos Reis, JR., Campinas (BR)

Correspondence Address: HELLER EHRMAN LLP, 4350 LA JOLLA VILLAGE DRIVE #700, 7TH FLOOR, SAN DIEGO, CA, 92122, US

(21) Appl. No.: 12/162,965

(22) PCT Filed: Aug. 1, 2006
(86) PCT No.: PCT/BR2006/000156

§ 371 Date: Jul. 31, 2008

(30) Foreign Application Priority Data
Mar. 17, 2006 (BR) PI 0601069-5

(51) Int. Cl. A61B 17/70

(52) U.S. Cl. 606/280; 606/289

(57) ABSTRACT

The present invention refers to a bone plate for the osteosynthesis of a fractured bone. According to an embodiment of the present invention, the bone plate comprises an upper side (101), intended to receive at least one bone screw and at least one fixation screw, and a lower side (102), intended to support itself on the bone, wherein said plate further comprises at least one pair (122) of holes (115, 116) distributed along a plane of the plate, and given that said pairs of holes (115, 116) consist of a first hole (115) intended to receive at least one bone screw, and a second hole (116) that has a thread, intended to receive at least one plate fixation screw, wherein said holes that comprise the pair (122) are tangent to each other and separated between their centers at a distance at least equal to the sum of the radiuses of said holes.

### FIELD OF THE INVENTION

The present invention generally refers to a bone plate for the osteosynthesis of a fractured bone.

More specifically, the present invention refers to a bone plate for the osteosynthesis of a fractured bone through which it is possible to accomplish a better fixation and anchoring of said bone plate, in comparison to the known plates of the prior art.

For the present specification, the terms “bone plate” or “plate for a bone” are employed indistinctly to designate a plate for the osteosynthesis of a fractured bone.

### BACKGROUND OF THE INVENTION

The prior art discloses various embodiments of bone plates intended for the osteosynthesis of a fractured bone. These plates may assume varied shapes, depending on the bone and type of fracture in which they are employed. However, in general, they have a flat shape with two sides and a series of holes in their surface, for the insertion of one or more bone screws, used for the fixation of the fracture, and one or more screws for the fixation of the plate on the fractured bone.

It is described, for example, in WO 97/09000, a bone plate that employs screws having a cone-shaped head made in a material that is harder than the material of the plate, to better anchor the screws. However, this type of fixation has the inconvenience of not being suitable for relatively thin plates, e.g. plates having with less than 2 mm of thickness. Moreover, this plate does not allow an angular adjustment of the screws, both for the bone screw and for the screw for fixation of the plate, which can be extremely deficient for the plate anchoring, depending on the type of the fracture of the bone.

On the other hand, WO 02/00127 discloses a bone plate that has at least one hole with an edge that projects toward the lower part of the plate. Although this type of plate solves the problem of the limitation of the thickness of the plate, it still has the inconvenient of not allowing an angular fixation of the screws to fix the plate on the bone.

There are also known in the state of the art bone plates with a series of pairs of holes, given that each pair is formed by a hole for the insertion of the bone screw and a hole with a thread for the insertion of the plate fixation screw, wherein the distance between the center of each hole is smaller than the sum of their radiuses, forming hence a hyperbola. This type of bone plate is described, for example, in the Brazilian patent application PI 0400679-8. Despite of solving the problem of the angling of the bone screws, the thread for placing the fixation screw does not follow the whole perimeter of the hole, which prevents good fixation of the screw, besides increasing the risk of stripping when fastened. Moreover, this type of bone plate does not allow the angling of the plate fixation screw, which limits the anchoring of the bone plate.

Notwithstanding, these plates of the state of the art generally have a cone-shaped thread in the hole, intended to receive the screw for the fixation of the plate, being that this screw for the fixation of the plate comprises in its head a correspondingly conical thread. Despite helping in plate fixation, the conical thread does not allow a good contact between the fillet of the thread and the screw, which can strip the thread when the screw is fastened. This is extremely costly, since it forces, at the time of the surgery, the removal of the plate already inserted for the insertion of a new plate.

In addition, the plates of the prior art normally present a bead in their ends. This is not advantageous when the fracture occurs in or near the end of the bone, since it prevents efficient fixation with the insertion of a bone screw, besides possibly interfering in the joint near the end of the bone.

Hence, it is one object of the present invention to provide a bone plate that overcomes the aforementioned inconveniences of the plates of the prior art.

More specifically, it is an object of the present invention a bone plate that allows the angling of screws for a better fixation of the plate.

Yet another object of the present invention is a bone plate that presents a thread profile that leads to a lower risk of stripping when a screw is fastened.

Finally, another object of the present invention is a bone plate that does not present a bead in its ends, so that it can be employed in the fixation of fractures that occur in the end of a bone.

### DESCRIPTION OF THE INVENTION

The above-mentioned objectives, among others, are achieved with the present invention, which consists in a bone plate that has an upper plane, intended to receive at least one bone screw and at least one fixation screw, and a lower plane, intended to support itself on a bone, in which said plate comprises at least one pair of holes laid out along at least one plane of the plate, wherein said pair of holes consists of a first hole intended to receive said at least one bone screw, and a second hole with a thread intended to receive said at least one fixation screw, characterized in that the holes of the pair are tangent to each other and separated between their centers at a distance at least equal to the sum of the radiuses of said holes, and in that the thread of at least one hole intended to receive a fixation screw is positioned at an angle in relation to a perpendicular axis of said plate.

Hence, according to a first aspect of the invention, the bone plate comprises the hole for bone screw and the hole for the plate fixation screw, separated at a minimal distance, equal to at least the sum of the radiuses of the holes, so that the thread of the hole intended to receive the plate fixation screw encompasses the entirely hole. Consequently, the plate fixation screw is better anchored, since its fillets are in contact with the thread fillets along the entire perimeter of the hole.

In a second aspect of the present invention, the thread of at least one hole intended to receive the plate fixation screw is inclined at an angle in relation to the perpendicular axis of the plate, so that the plate fixation screw is not introduced and fixated perpendicularly in relation to the plate, but rather at a certain angle. This characteristic allows a better anchoring of the plate on the bone. The inclination angle of the thread of the hole intended to receive the plate fixation screw may vary, and shall be comprised between 1° and 10°, particularly between 3° and 7°, and more particularly approximately 5°.

According to a particular embodiment of the invention, when there is more than one pair of holes, the threads of the holes intended to receive the plate fixation screws are inclined in an alternated manner, and can be interpolated or followed by a thread that is perpendicular to the plate. That is, a first hole intended to receive the plate fixation screw can be at a first inclination in relation to the perpendicular axis of the plate, a second hole intended to receive the plate fixation screw can be at an opposing inclination in relation to the perpendicular axis of the plate, and a third hole can be centered perpendicularly in relation to the perpendicular axis of the plate, and henceforth. The inclination sequence of the holes may vary, as long as it is alternated. This characteristic confers a better anchoring of the plate on the bone, since the forces exerted in the fixation of the plate are distributed in many points on the bone, and not only along the longitudinal axis of the plate. The distribution of forces along the bone becomes more balanced, therefore avoiding that the bone splits when the bone plate is fixated, besides reinforcing the anchoring of the bone on the plate.

At least one pair of holes must be contained according to a plane of the plate. The term “according to one plane of the plate” shall be interpreted as being at least one pair of holes distributed on at least one plane of the plate. If there is more than one pair of holes, they may be distributed on the same or in different planes, depending on the shape of the plate. Alternatively, if there is more than one pair of holes, they can be distributed longitudinally from one plate axis, aligned or not. The spacing between the pair of holes, if there is more than one, may also vary according to the format and the size of the plate.

The thread of the holes intended to receive the plate fixation screws, according to a particular embodiment of the invention, has a hyperbolic transversal profile. This configuration of the thread allows a better contact between the thread fillets on the hole and the thread fillets on the screw, hence avoiding stripping the screw when it is fastened. Likewise, the plate fixation screw shall comprise on its head a corresponding thread with a hyperbolic shape. The diameter of the hole and the pitch of the thread may also vary depending on the size of the plate and its application.

The hole intended to receive the bone screw may comprise an oblong shape, as occurs in the plates of the prior art, so as to allow a free angling of the bone screw on the plate, to adapt it in different types of fractures.

According to another particular embodiment of the invention, the bone plate can also eliminate the borders in its ends. This characteristic is highly advantageous when one wishes to fixate a fracture that occurred on the end of a bone. This is because the borders of the prior art plates makes difficult the insertion of the bone screw, and may also interfere in the joint near the bone.

### BRIEF DESCRIPTION OF THE DRAWINGS

The present invention will now be described in relation to a particular embodiment, referring to the attached figures. Such figures are schematic, and their dimensions and proportions may not correspond to reality, once they are intended to illustrate the present invention in a didactic form without imposing any limitative character except that defined by the attached claims, wherein:

FIG. 1 is a top view of a bone plate according to an embodiment of the present invention;

FIG. 2 is a cross-section lateral view of the plate of FIG. 1;

FIG. 3 is a bottom plane view of the plate of FIG. 1;

FIG. 4 is a front view according to the detail B of plate of FIG. 1; and

FIG. 5 is a front view according to the cross-view AA of the plate of FIG. 1.

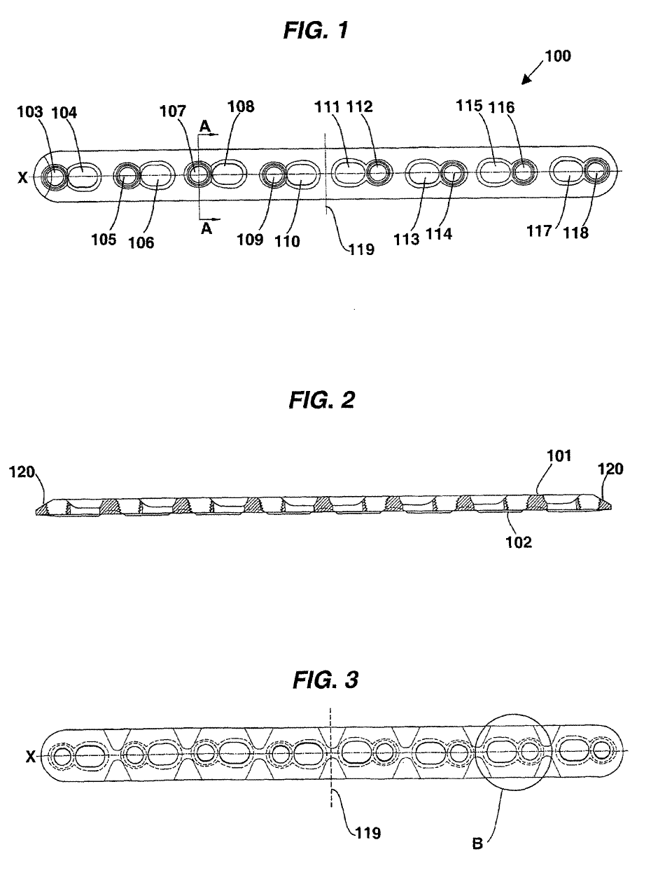

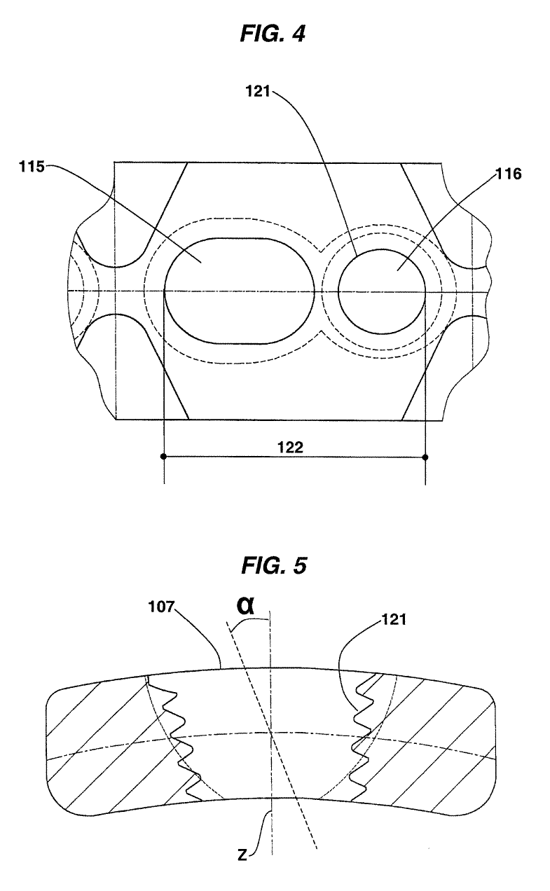

### DESCRIPTION OF PARTICULAR EMBODIMENTS

On the figures, a bone plate is represented according to a particular embodiment of the present invention. The numerical references are repeated to indicate equal or similar elements in the different figures.

The bone plate (100) represented in the figures according to this embodiment has an upper plane (101), which is intended to receive the bone screws and the plate fixation screws, and a lower plane (102), intended to support itself on the bone. The profile of both upper (101) and lower (102) planes can be slightly curved, for a better adaptation on the bone of a vertebrate being, as can be seen in the figures. This curved profile may be comprised within a radius of approximately 10 mm and 50 mm, particularly between 15 mm and 25 mm.

The bone plate, as represented, comprises eight pairs of holes (103 to 118).

Each pair (122) of holes comprises one hole that corresponds to a thread (103, 105, 107, 109, 112, 114, 116, 118), intended to receive a screw (not shown) for the fixation of the plate, and an oblong hole (104, 106, 108, 110, 111, 113, 115, 117) that will serve to receive the bone screw (now shown).

According to the present invention, the bone plate must comprise at least one pair (122) of holes. However, the number of pairs of holes may vary depending on the size and dimensions of the plate. For example, for applications in larger bones, such as the femur, a plate with larger dimensions may be employed. However, for smaller bones, such as the radius or cubitus, a smaller plate shall be used. A number of pairs (122) of holes particularly adequate is comprised between 2 and 20, particularly between 6 and 16 pairs of holes.

Likewise, the distribution of the pairs of holes (122) may vary according to the shape of the plate. Certain specific applications require plates with “T” or “L” shapes or even other. Hence, according to the present invention, it is necessary that the bone plate (100) comprises at least one pair of holes (122) distributed on a plane of the plate.

According to the particular embodiment described herein, the pairs of holes (103 to 118) are divided in equal number in relation to the center of the plate (119) and are distributed along the plane on the longitudinal axis (X) of the plate. According to this embodiment, there are 4 pairs of holes substantially aligned along the longitudinal axis (X) of the plate and equally divided for each plane starting from the center (119) of the plate (100). However, the pairs (122) of holes do not need to be aligned, and may be spaced from each other. The distance between the pairs of holes may also vary with the size and shape of the plate.

Yet according to the particular embodiment herein described, the pairs of holes are positioned so that the holes intended to receive the bone screw (104, 106, 108, 110, 111, 113, 115, 117) are facing toward the center of the plate (119). However, the pairs of holes can be oriented in any direction.

The holes intended to receive the bone screw (104, 106, 108, 110, 111, 113, 115, 117) are preferably oblong holes, to grant a certain level of freedom in the fixation of the bone screw on the fractured bone. This oblong shape allows the bone screw to be fixated at an angle in relation to the plate (100), when necessary, hence granting a better contact in relation to the fracture.

According to a first aspect of the present invention, the pairs of holes comprise one hole to receive the bone screw (104, 106, 108, 110, 111, 113, 115, 117) and one hole to receive the plate fixation screw (103, 105, 107, 109, 112, 114, 116, 118), and said holes are tangent to each other. The distance between the centers of the holes is at least equal to the distance of the sum of the largest radius of the holes (103 to 118).

Therefore, contrary to bone plates of the prior art, the thread (121) of the holes (103, 105, 107, 109, 112, 114, 116, 118) intended to receive the fixation screw encompasses the entire perimeter of the holes. Thus, according to this characteristic of the bone plate of the invention, there is a better fixation and anchoring of the plate fixation screw, since the fillets of the fixation screw are in integral contact with the thread of the holes (103, 105, 107, 109, 112, 114, 116, 118).

Additionally, according to a particular aspect of the invention, at least one thread (121) of the holes (103, 105, 107, 109, 112, 114, 116, 118) intended for the fixation screw is inclined in relation to the perpendicular axis (Z) of the plate (100). This inclination feature grants a greater stability of fixation of the plate on the bone, since the load is distributed along various points, and not just on the perpendicular plane (Z). Particularly, the inclination of the holes is alternated starting from the center (119) of the plate, with holes with the thread inclined to the right in relation to the perpendicular axis (Z) of the plate, with holes with the thread inclined to the left in relation to the perpendicular axis (Z), and with perpendicular holes, that is, without thread inclination.

For example, if a plate has four pairs of holes, the inclination of the holes can be alternated in relation to the center of the plate. In this case, the hole with thread (109) has an inclination to the right in relation to the perpendicular axis (Z) of the plate; the hole with thread (107) has an inclination to the left in relation to the perpendicular axis (Z) of the plate. Likewise, the hole with thread (112) has an inclination to the left in relation to the perpendicular axis (Z) of the plate; and the hole with thread (114) has an inclination to the right in relation to the perpendicular axis (Z) of the plate. In this case, there are no holes with a perpendicular thread in relation to the plate.

However, according to the plate herein described, the holes with thread (112, 118 and 107) have an inclination to the right in relation to the perpendicular axis (Z) of the plate, the holes with thread (114, 103 and 109) have an inclination to the left in relation to the perpendicular axis (Z) of the plate, and the holes with thread (105, 116) are perpendicular to the plate.

This relationship can alternate, with perpendicular holes between inclined holes, for example, as long as there are no two consecutive holes with the same inclination.

Particularly, the angle of inclination (a) of the holes with thread must be the same for all holes, respecting the direction of inclination and the holes whose thread does not have an inclination, when present. The angle of inclination (a) shall be comprised between 1° and 10°, particularly between 3° and 7°, and more particularly approximately 5°

Advantageously, this alternation of inclination between the threads of the holes intended to receive the plate fixation screw grants a better fixation and anchoring of the plate on the bone, since the load is distributed along various points on the bone, avoiding, hence, its rupture and/or loosening.

Particularly, the thread (121) of holes (103, 105, 107, 109, 112, 114, 116, 118) intended to receive the plate fixation screw must have a hyperbolic transversal profile. This characteristic allows a better contact between the screw fillets (not shown) and the thread fillets, thus avoiding stripping the screw when fastened to the plate (100).

The size of the holes and the dimensions of the plate, such as length, width, and thickness may vary depending on the application needs.

The plate (100) also comprises a chamfer (120) that eliminates the beads applied on the plates of the prior art. The chamfer may be accomplished in interference with the holes closer to the ends (103, 118), with no damage to the fixation of the fixation screw on the plate. The length of the chamfer may be comprised between approximately 2 and 6 mm, and the inclination between approximately 15° and 60°.

Therefore, according to the embodiment of a bone plate here illustrated, it is evident that the inconveniences of bone plates found in the prior art are eliminated. This occurs because, according to a first feature of the present invention, the holes (103, 105, 107, 109, 112, 114, 116, 118) intended to receive the plate fixation screw have a thread that goes around the entire perimeter, so as to better anchor the screw, and hence, the plate. Notwithstanding, the hole inclination, in particular an alternate inclination, allows the plate fixation screws to be supported on various points on the bone, better distributing the load and hence, avoiding its rupture.

According to another feature of the present invention, the thread (121) of the holes (103, 105, 107, 109, 112, 114, 116, 118) intended to receive the plate fixation screw has a hyperbolic profile, which allows better contact between the hole fillets and the screw fillets, hence avoiding that the screw strips during fastening. Finally, the bone plate of the present invention, according to another feature, eliminates the beads of its ends, using a chamfer, hence allowing its use in fractures on bone ends.

It is important to comprehend that, although the present invention has been described in relation to a particular embodiment, those skilled in the art may develop many variations of structural details and expand the aforementioned invention for other types of applications, without, however, deviating from the principles of the invention. For example, the various elements shown in the particular embodiments can be combined in a manner not illustrated in the drawings. Hence, the claims annexed hereto must be interpreted as covering all the equivalents that can be framed within the ambit and character of the invention.

1. Bone Plate (100) comprising an upper plane (101), intended to receive at least one bone screw and at least one fixation screw, and a lower plane (102), intended to support itself on a bone, in which said plate comprises at least one pair (122) of holes (103-118) laid out along at least one plane of the plate (100), wherein said pair of holes (103 to 118) consists of a first hole (104, 106, 108, 110, 111, 113, 115, 117) intended to receive said at least one bone screw, and a second hole (103, 105, 107, 109, 112, 114, 116, 118) with a thread intended to receive said at least one fixation screw, characterized in that the holes of the pair (122) are tangent to each other and separated between their centers at a distance at least equal to the sum of the radiuses of said holes, and in that the thread (121) of at least one hole (103, 105, 107, 109, 112, 114, 116, 118) intended to receive a fixation screw is oriented at an angle (α) in relation to a perpendicular axis (Z) of said plate.

2. The Bone Plate of claim 1, wherein when there is more than one hole intended to receive the plate fixation screw, the thread (121) of the holes (103, 105, 107, 109, 112, 114, 116, 118) is inclined in a alternated direction in relation to the perpendicular axis (Z) of the plate without two consecutive holes having the same direction of inclination.

3. The Bone Plate of claim 1 or 2, wherein it further comprises holes intended to receive the plate fixation screw whose thread (121) follows the perpendicular axis (Z) of the plate.

4. The Bone Plate of claim 1, wherein said angle (α) is comprised between approximately 1° and 10°, particularly between 3° and 7°, and more particularly approximately 5°.

5. The Bone Plate of claim 1 or 4, wherein the angle (α) is equal for all holes (103, 107, 109, 112, 114, 118) that have an inclination, even though the direction of inclination is different.

6. The Bone Plate according to one of the previous claims, wherein said thread (121) has a hyperbolic transversal profile.

7. The Bone Plate according to one of the previous claims, wherein said hole (104, 106, 108, 110, 111, 113, 115, 117) intended to receive the bone screw is an oblong hole.

8. The Bone Plate according to one of the previous claims, wherein it comprises 2 to 20 pairs of holes, particularly 6 to 16 pairs of holes.

9. The Bone Plate according to one of the previous claims, wherein the pairs of holes are oriented so that the holes intended to receive the bone screw (104, 106, 108, 110, 111, 113, 115, 117) are turned toward the center (119) of the plate.

10. The Bone Plate according to one of the previous claims, wherein the pairs of holes (122) are substantially aligned along the plane on a longitudinal axis (X) of the plate.

11. The Bone Plate according to one of the claims 1 to 9, wherein the pairs of holes (122) are substantially misaligned with each other, although distributed on the plane according to a longitudinal axis (X) of the plate.

12. The Bone Plate according to one of the previous claims, wherein it comprises a chamfer (120) on its ends.

13. The Bone Plate according to claim 12, wherein the chamfer is accomplished in interference with the holes closer to the ends (103, 118) of the plate.

14. The Bone Plate according to claim 13, wherein said chamfer has a length of approximately 2 to 6 mm, and an inclination of approximately 15° to 60°.

15. The Bone Plate according to one of the previous claims, wherein the profile of the upper (101) and lower (102) planes is curved, whose radius is comprised between approximately 10 mm and 50 mm, particularly between approximately 15 and 25 mm.

16. The Bone Plate according to one of the previous claims, wherein its shape is in “T” or “L”.
